(19) United States
(12) Patent Application Publication
(10) Pub. No.: US 2009/0069854 A1
(43) Pub. Date: Mar. 12, 2009

(54) Implantable electric device

(75) Inventors: Yaron Keidar, Haifa (IL); Michal Reshef, Haifa (IL)

Correspondence Address: MARTIN D. MOYNIHAN d/b/a PRTSI, INC., P.O. BOX 16446, ARLINGTON, VA, 22215, US

(73) Assignee: Eqlibra Medical Ltd., Kiryat-Shmona (IL)

(21) Appl. No.: 11/918,992

(22) PCT Filed: Apr. 2, 2006
(86) PCT No.: PCT/IL06/00418

§ 371 Date: Oct. 22, 2007

Related U.S. Application Data
(60) Provisional application No. 60/673,386, filed on Apr. 21, 2005.

(51) Int. Cl. A61N 1/362; B23K 26/36; A61M 1/12; A61F 2/82

(52) U.S. Cl. 607/3; 219/121.72; 29/557; 600/16; 623/1.15

(57) ABSTRACT

An implantable electric device comprising: a deployable round structure initially in a cramped state and adapted to be expanded into a deployed state, with at least one of a plurality of coils provided about the structure; a power source; and an electric circuitry for generating alternating currents in said at least one of the plurality of coils to generate an alternating electromagnetic field within the structure. In some embodiments of the invention it serves as a motor, and in some embodiments it serves to enhance blood flow within a patient's vasculature.

### FIELD OF THE INVENTION

The present invention relates to implantable devices. More specifically, the present invention relates to implantable electric devices, making use of alternating magnetic fields, for deployment inside a patient's body.

### BACKGROUND OF THE INVENTION

Exiting devices, such as stents, improve blood flow by increasing the cross-section area of blood vessels in places where clogging or narrowing of a blood vessel restricts the flow. Other devices such as cardioplesia pumps and left-ventricular-assist-devices, for example, can be inserted surgically and pump large volumes of blood. The present invention introduces a miniature pump incorporated in a stent that can be deployed using trans-catheter deployment, in a standard catheterization procedure. This pump can only pump small volumes of blood, and is intended to run for a period ranging from a few days to a few weeks, and treat a local target tissue or organ.

Several blood-flow assist devices were patented in the past. U.S. Pat. No. 6,210,318 (Lederman) discloses a balloon pump system including catheter-mounted pumping balloon configured to be positioned within a desired body passageway to pump a fluid through the body passageway. A stent is percutaneously deployed within the body passageway. The pumping balloon is percutaneously deployed within the stent such that the stent is interposed between the pumping balloon and the walls of the body passageway. The stent limits the compliance of the body passageway, preventing the passageway in the vicinity of the pumping balloon from significantly expanding or contracting in response to forces generated by inflation and deflation of the pumping balloon. As a result, a volume of fluid substantially equivalent to a change in volume of the pumping balloon is displaced when the pumping balloon is inflated or deflated. The stent improves the efficiency of the balloon pump by providing anchorage, and by limiting the compliance of the blood vessel (mainly aorta), in the pumping region.

In U.S. Pat. No. 5,749,855 (Reitan) implantable catheter pumps are disclosed including a drive cable, with one end of the drive cable being connectable to a drive source, a collapsible drive propeller at the other end of the drive cable, with the collapsible drive propeller being adjustable between a closed configuration in which the collapsible drive propeller is collapsed upon the drive cable and an open configuration in which the collapsible drive propeller is expanded so as to be operative as an impeller, and a sleeve extending between one side of the collapsible drive propeller and the other side of the collapsible drive propeller with the sleeve being movable between configurations in which the collapsible drive propeller is in the open and closed configuration.

US 2003/0135086 (Khav et al.) discloses an inflatable circulation assist device consisting of an inflatable stator housing an impeller with inflatable blades of varying shapes and sizes. The invention is introduced into the patient percutaneously. The circulation assist device is a small pump packaged into a compact form that is attached to a long flexible driveshaft. The pump is inserted along a guidewire to a target location, and then the pump is inflated. The circulation assist device's exterior is designed to expand only so much as to closely fit whatever cardiovascular system element in which it is placed for operation. The vascular assist device can be expanded either by inflation with a fluid. The driveshaft, which connects to the circulation assist device's impeller and extends outside the patient's body, is rotated by an external motor. After the circulation assist device is no longer needed, it is collapsed into a compact form and removed from the patient percutaneously.

U.S. Pat. No. 6,176,848 (Rau et al.) discloses a blood pump having a motor housing and a pump housing which are rigidly connected to one another in an axially spaced relationship. Both housings are of substantially the same diameter and are sized to enable the pump to be introduced via catheter through the body's blood-vessel system. The impeller is mounted in the pump housing on a longitudinally and radially acting bearing designed as a point-support bearing. To avoid oscillation of the impeller, it is fitted with an alignment device which may have a hydrodynamic or mechanical action. Rotation of the motor is transferred to the impeller via a magnetic coupling.

In U.S. Pat. No. 5,290,227 (Pasque) a pump which is well-suited for such implantation is disclosed, having an impeller design that generates central axial flow (CAF). After implantation of the CAF pump, blood flows through the hollowed-out rotor shaft of an electric motor. The device acts, in fact, as a turbine.

U.S. Pat. No. 5,503,615 (Goldstein) discloses an implantable ventricular assist device, which has only one moving part. This part consists of a conical rotor with vanes which spiral upward from the base in a direction opposite to the direction of rotation. There are no valves within the device itself, but one or two valves are situated in the conduits connected to it. The device is powered by a constant running electric motor which screws into the base of the rotor housing. The motor is connected to a portable external battery by means of subcutaneous electrical leads.

U.S. Pat. No. 5,879,375 (Larson et al.) describes a surgically implantable reciprocating pump employing a check valve as the piston, which is driven by a permanent magnet linear electric motor to assist either side of the natural heart. The pump is implanted in the aorta or pulmonary artery using vascular attachment cuffs such as flexible cuffs for suturing at each end with the pump output directly in line with the artery. The pump is powered by surgically implanted rechargeable batteries. In another embodiment, pairs of pumps are provided to replace or assist the natural heart or to provide temporary blood flow throughout the body, for example, during operations to correct problems with the natural heart.

U.S. Pat. No. 6,217,541 (Yu) discloses a blood pump comprising a cross-flow pump head having an elongated generally cylindrical housing portion. The housing portion defines a blood inlet port on a surface thereof and a blood outlet port on an opposite surface thereof. An impeller within the housing portion provides cross-flow of the blood from the inlet port around and/or across the rotational axis of the impeller to the outlet, and a motor is provided for driving the cross-flow pump head. The blood pump may be small enough to permit percutaneous insertion of the pump into a patient's blood vessel, and thus may be utilizable as a left ventricular assist device. To this end, a collapsible polymeric outflow tube is coupled to the blood flow outlet and is adapted for directing the blood from the left ventricle to the aorta through the aortic valve.

In U.S. Pat. No. 6,527,699 (Goldowsy) a non-contact axial flow turbo blood pump for propelling blood is disclosed, which is composed of a pump housing that defines a pump axis, with inlet, outlet openings at opposite axial ends of the pump housing, a rotor unit that defines a rotor axis, and opposing rotor axial ends. The pump magnetically suspends the rotor within the pump housing at the rotor axial ends so as to avoid causing physical contact between the housing to define fluid gaps between the rotor axial ends, and the magnetic suspension elements.

U.S. Pat. No. 5,089,016 (Millner et al.) describes a blood pump having a toroidal shaped chamber concentrically positioned around a cylindrically shaped hydraulic pump. The toroidal chamber has two toroidal shaped portions, one portion having a substantially rigid external wall and the other having an external wall formed of a flexible membrane. The chamber has an inlet and output port suitable for connection to a blood flow supply. The flexible wall portion of the toroidal chamber is enclosed within a hydraulic chamber, fluidically coupled to the hydraulic pump. The hydraulic pump is controlled so that an increase of pressure in the hydraulic chamber results in a decrease of volume in the toroidal chamber, thus providing for pumping of the blood through that portion of the chamber. The toroidal shape provides for optimal non-coagulating flow, while the rigid wall of the chamber, together with the flexible membrane provide for membrane motion along only one axis, normal to the circumference of the toroid, preventing damage to the membrane. The blood flow inlet port is positioned to direct fluid in a tangential direction against the perimeter wall of the toroid.

U.S. Pat. No. 5,643,172 (Kung et al.) discloses a circulatory apparatus for assisting the movement of fluids with a pulsatile flow. The circulatory apparatus includes a restrictable tube, an element for passively restricting a segment of the tube, two or more elements for selectively restricting segments of the tube, and a controller for directing selective restricting elements. The controller directs restricting elements in a manner that provides a cyclical pulsatile pattern of restriction along the length of the tube.

U.S. Pat. No. 6,942,611 (Siess) discloses a paracardiac blood pump designed for protruding through the cardiac wall into the heart with a portion of its housing and for suctioning blood from the heart. The blood is pumped into one of the blood vessels connected with the heart through a line that extends outside the heart. A cannula is arranged in front of the inlet of the pump ring. The housing and the cannula have approximately the same outer diameters of 13 mm at most. The housing, together with the cannula, can thus be inserted into the heart through a puncture hole that is produced in the cardiac wall without removing material.

It is an object of the present invention to provide a novel blood-flow assist device.

Another object of the present invention is to provide such a trans-catheter device for enhancing blood flow, incorporated with a stent.

Another object of the present invention is to provide a method of fabricating miniature collapsible blood flow assist devices.

Another object of the present invention is to provide a method for deploying one or more such assist devices in a patient, and retrieve them at a later date.

### SUMMARY OF THE INVENTION

There is thus provided, in accordance with some preferred embodiments of the present invention, an implantable electric device comprising:

a deployable round structure initially in a cramped state and adapted to be expanded into a deployed state, with at least one of a plurality of coils provided about the structure;

a power source; and

an electric circuitry for generating alternating currents in said at least one of the plurality of coils to generate an alternating electromagnetic field within the structure.

Furthermore, in accordance with some preferred embodiments of the present invention, the device is further provided with a separately deployable element for deployment within the structure in the deployed state, having at least one of a plurality of ferromagnetic elements, the element adapted to be actuated by the alternating magnetic field.

Furthermore, in accordance with some preferred embodiments of the present invention, said at least one of the plurality of ferromagnetic elements comprises at least one of a plurality of magnetic elements.

Furthermore, in accordance with some preferred embodiments of the present invention, the separately deployable element comprises a rotor.

Furthermore, in accordance with some preferred embodiments of the present invention, the rotor comprises a propeller.

Furthermore, in accordance with some preferred embodiments of the present invention, the propeller comprises a plurality of flaps provided with a lateral twist, coupled to the separately deployable element peripherally and capable of switching between two states, a first state being when the flaps are aligned with the structure leaving free space within the structure and a second state being when the flaps are folded forming a propeller.

Furthermore, in accordance with some preferred embodiments of the present invention, the flaps each comprise a looped wire and a polymeric surface.

Furthermore, in accordance with some preferred embodiments of the present invention, the ferromagnetic elements comprise magnetic elements embedded peripherally in loops of the wire.

Furthermore, in accordance with some preferred embodiments of the present invention, the flaps are made of metal.

Furthermore, in accordance with some preferred embodiments of the present invention, the structure and the rotor are made from a shape memory alloy.

Furthermore, in accordance with some preferred embodiments of the present invention, beads are provided on the rotor serving as bearings.

Furthermore, in accordance with some preferred embodiments of the present invention, there is provided a method for providing alternating electromagnetic fields within a lumen in a body of a patient, the method comprising:

providing an implantable electric device comprising:

a deployable round structure initially in a cramped state and adapted to be expanded into a deployed state, with at least one of a plurality of coils provided about the structure, a power source; and an electric circuitry for generating alternating currents in said at least one of the plurality of coils to generate an alternating electromagnetic field within the structure;

deploying the deployable round structure within the lumen using a catheter.

Furthermore, in accordance with some preferred embodiments of the present invention, the method further comprises deploying a separately deployable element within the structure in the deployed state, having at least one of a plurality of ferromagnetic elements, the element adapted to be actuated by the alternating magnetic field.

Furthermore, in accordance with some preferred embodiments of the present invention, the rotor comprises a propeller comprising a plurality of flaps provided with a lateral twist, coupled to the separately deployable element peripherally and capable of switching between two states, a first state being when the flaps are aligned with the structure leaving free space within the structure and a second state being when the flaps are folded forming a propeller; the method further comprising actuating the alternating electromagnetic fields causing the propeller to rotate in the second state.

Furthermore, in accordance with some preferred embodiments of the present invention, the lumen comprises a blood vessel, the device being used to enhance blood flow.

### BRIEF DESCRIPTION OF THE DRAWINGS

In order to better understand the present invention, and appreciate its practical applications, the following Figures are provided and referenced hereafter. It should be noted that the Figures are given as examples only and in no way limit the scope of the invention. Like components are denoted by like reference numerals.

FIG. 1 illustrates an exploded view of a deployable blood flow enhancing device in accordance with a preferred embodiment of the present invention.

FIG. 2 illustrates a deployable wire turbine for a deployable blood flow enhancing device in accordance with a preferred embodiment of the present invention.

FIG. 3 illustrates a deployable blood flow enhancing device in accordance with a preferred embodiment of the present invention, the deployable turbine seen inside.

FIG. 4 illustrates an alternative embodiment for a deployable turbine for a blood flow enhancing device in accordance with a preferred embodiment of the present invention, in a contracted state.

FIG. 5 illustrates the deployable turbine shown in FIG. 4 in a deployed state.

FIG. 6 illustrates the deployable turbine shown in FIG. 5 with the flaps in operational orientation.

FIG. 7 illustrates a deployable blood flow enhancing device in accordance with a preferred embodiment of the present invention, with a control unit.

FIG. 8 illustrates a deployable blood flow enhancing device in accordance with a preferred embodiment of the present invention, with a control unit, implanted in a patient.

FIG. 9 illustrates a first stage in the manufacturing process of a deployable turbine for a deployable blood flow enhancing device in accordance with a preferred embodiment of the present invention.

FIG. 10 illustrates a second stage in the manufacturing process of a deployable turbine for a deployable blood flow enhancing device in accordance with a preferred embodiment of the present invention, where the flaps are press-folded to form a turbine.

FIG. 11 illustrates a magnetic element to be incorporated with a deployable turbine for a blood flow enhancing device in accordance with a preferred embodiment of the present invention.

FIG. 12 illustrates a turbine propeller with embedded magnetic elements.

FIG. 13 illustrates a first step in incorporating electric motor coils with a stent.

FIG. 14 illustrates a second step in incorporating electric motor coils with a stent.

FIG. 15 illustrates a third step in incorporating electric motor coils with a stent.

FIG. 16 illustrates a fourth step in incorporating electric motor coils with a stent.

FIG. 17 illustrates a fifth step in incorporating electric motor coils with a stent.

FIG. 18 illustrates the wire turbine shown in FIG. 2 with beads threaded on the wire.

### DETAILED DESCRIPTION OF PREFERRED EMBODIMENTS

The present invention introduces an implantable electric device that is capable of generating alternating electromagnetic fields. In a preferred embodiment of the present invention the implantable electric device serves as a deployable blood flow enhancing device that incorporates a electromagnetic turbine with a stent. In other embodiments of the present invention, the implantable electric device is used as a motor for other purposes, such as for example, a drill, an electrically operated valve, and others. By “motor” it is meant, in the context of the present invention, any design of an electric device that has a first fixed element and a second movable element that is moved by electromagnetic forces exerted between the two elements of the motor. This can be for example a rotary motor, a solenoid where the core inside the coil is moved back and forth, etc.

The device, according to some preferred embodiments of the present invention, is practically an electric pump that can be deployed in an artery or a vein and is deployable using trans-catheter methods.

The device of the present invention has the advantage of possessing a low profile in its contracted state, allowing it to be deployed using a catheter or similar deployment tool. This means that the device of the present invention can be deployed in a blood vessel in minimally invasive manner, without having to perform a major medical procedure on the patient.

Basically, a deployable blood flow enhancing device according to a preferred embodiment of the present invention comprises three main parts: An expandable stent frame, an expandable turbine propeller and a controller unit.

Some medical conditions result in blood vessels with blood flow too poor to supply the target organs with enough oxygen and nutrients for healthy and proper function. The blood pump according to the present invention has a unique design, is very small and is designed for small blood vessels. The deployment of the blood pump is intended to be performed using a catheter. The device of the present invention has three main parts: A controller box, an outer frame and revolving turbine propeller. The controller includes an electric battery (or other power source), and an electric circuit. The battery serves as a power source to power the pump. The electric circuit powered by the battery, generates one or more alternating current signals to drive the electric motor of the pump. The alternating current signals are carried by wires to electric coils distributed around the frame. The frame is a structure that forms the outer enclosure of the pump. Since the pump is intended to be placed in a small blood vessel, and is inserted to that blood vessel by a catheter, the frame is a collapsible and expandable stent. The frame is initially cramped and mounted on a catheter outside of a patient's body. The catheter is inserted to the patient's vascular system and advanced to the target location for the pump to be deployed. The frame is expanded to the diameter of the blood vessel using one of several deployment methods (see explanation hereinafter). The function of the frame is to be a conduit for the blood flow (and hold the blood vessel open, as traditional stents do) and to constrain the turbine in a way that allows the turbine to revolve freely, but hold it from migrating distally or proximally. An additional function of the frame is to serve as the stator of the electric motor formed by the combination of the frame and the turbine. To accomplish that, electric coils are distributed around the frame. Lead wires from the electric circuit in the controller box, conduct the alternating current signals to the coils. The current going through the coils generates an electromagnetic field. The arrangement of the coils around the frame and the phase delay from one alternating current signal to the next that is maintained by the controller box, generate a revolving electromagnetic field in the lumen of the frame, much like in standard electric motors. The turbine comprises also a round and initially hollow structure, with magnets and propeller blades mounted on it. The purpose of the turbine is to revolve inside the frame and push the blood flowing through it. The magnets are arranged all around the turbine, and when placed in the revolving magnetic field inside the frame will cause the turbine to revolve. The blades are mounted all around the turbine forming a propeller, with the blades initially substantially parallel to the direction of the flow, but capable of folding into a propeller formation during operation. In some other embodiments of the present invention the propeller formation is fixed. The revolutionary motion of the turbine causes the blades to push blood through the pump creating or increasing blood flow. Since the pump is intended to be placed in a small blood vessel, and is preferably inserted to that blood vessel by a catheter, the turbine is also designed as a collapsible and expandable structure. The turbine, in some preferred embodiments of the present invention is separately cramped and mounted on a catheter outside of a patient's body. The catheter is inserted to the patient's vascular system and advanced to the target location where the frame is already deployed. The turbine is expanded to the proper diameter to engage with the frame. The turbine can be retracted from its deployed location by a catheter at a later time, once it has served its purpose. The controller box and lead wires can also be detached from the frame and removed. The frame, however, can remain permanently in the patient's body serving to hold the blood vessel open, just like an ordinary stent.

Reference is now made to the accompanying figures.

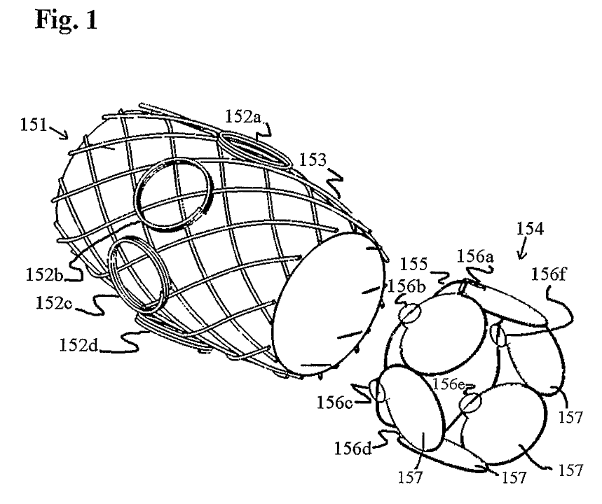

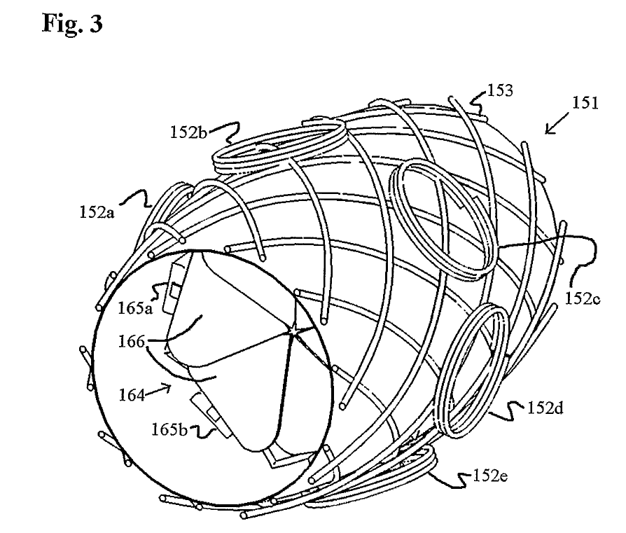

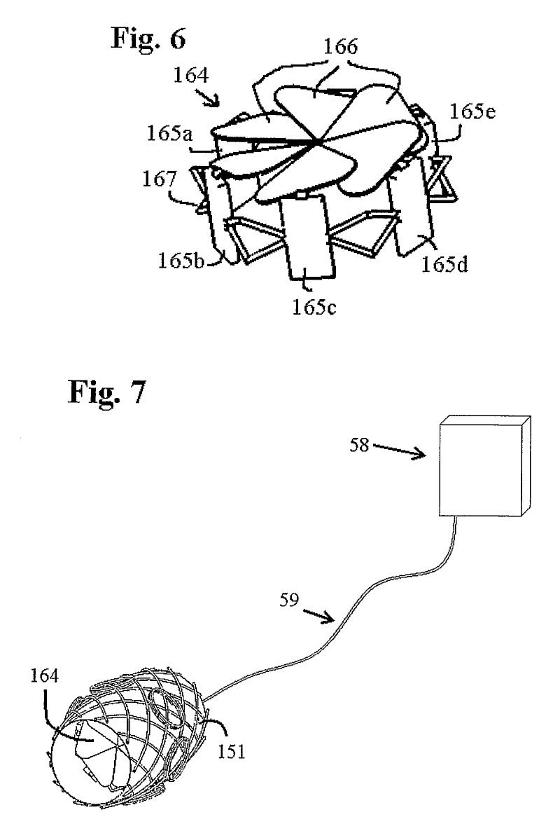

FIG. 1 illustrates an exploded view of a deployable blood flow enhancing device in accordance with a preferred embodiment of the present invention. An expandable frame 151 preferably made from a wire frame 153 is embedded with two or more electric wire coils 152a, 152b, 152c, 152d. Running alternating current through the coils generates a revolving magnetic field that causes the propeller turbine 154 (made too from a wire frame 155) to rotate. In one embodiment the frame is cylinder shaped. In another embodiment the frame is barrel shaped. The propeller turbine 154 comprises a plurality of flaps 157, that when folded (see FIG. 3 and FIG. 6) present orientation in an inclined position with respect to the direction of the flow, in a blade formation that when rotated exerted hydrodynamic forces on the flow forcing it in the desired direction. The propeller turbine is equipped with a plurality of magnetic elements (156a, 156b, 156c, 156d, 156e) arranged about the perimeter of the turbine. The coils generate an alternating magnetic field (due to the alternating currents that pass through the coils), causing the turbine to revolve. The flaps can preferably be made from metal or a thin polymer membrane stretched over a wire frame of a wire loop to create a surface. To make flaps from metal wire with thin polymer membrane, the wire is first formed in the desired shape of the turbine. The wire is shaped into closed loops which will become the frames of the flaps. The wire frame is attached to the magnetic elements, and than it is dipped in liquid polymer. The polymer forms a thin film in the wire loops and than dries and hardens to form the flaps. This is not the only way to form flaps and is described by way of example only. The contour of the turbine, once expanded, is shaped to match the frame, in such a way that it resides in the middle of the frame, and can rotate freely but cannot move proximally or distally in the frame, as the frame is shaped in a barrel form, or has other means to limit the migration of the turbine (for example, using protrusions inside the frame that prevent the turbine from leaving the frame). To be expandable, the turbine propeller is also built of a metal wire frame. The frame can be self-expanding, for example, a memory shape alloy such as Nitinol™, or deployed and expanded by a balloon. One or more magnets are attached to the turbine to drive the propelling motion of the turbine. The blades of the propeller are attached to the frame (or to the magnets) in a flexible elastic way. One embodiment is to have the blades mounted parallel to the tubular frame and the walls of the blood vessels, keeping the lumen open while the turbine is standing still. When the pump is started and the blades fold inwardly by the pressure of the blood they are pushing the other way. This allows the pump to be switched on and off periodically, without too much obstruction to the flow when the pump is not operating.

The frame is deployed first, (it may be made from a memory shape alloy such as Nitinol that deploys at a predetermined temperature, or may be delivered and deployed by a balloon. The electric cable leading to the frame may serve as a guide wire to deploy the propeller, and may be used later (days or weeks later) to retrieve it. After the deployment of the propeller, the controller box is attached to the proximal end of the lead wires cable, the power is turned on, and the pump is activated. When the pump is inactivated the controller is detached, the propeller is retrieved, and the electric cable is snapped close to the frame. The frame is left behind permanently like a stent.

A deployable blood flow enhancing device in accordance with a preferred embodiment of the present invention may be provided in several general shapes. One alternative shape is in the form of a cylinder. Another alternative shape is in the form of a barrel (as shown in the figures). Other shapes are possible too.

Some embodiments of the device of the present invention can be incorporated with an implantable pace maker or a stimulator, sharing the housing for the electric circuit and battery and using one lead wire for the purpose of stimulating the heart and driving the pump. Another embodiment of the device of the present invention, the structure of the device is coated with anti-coagulation agent such as Heparin to prevent blood from clotting on the device and obstructing blood flow or pump action.

Other tools may be used in the pump implantation procedure, for example, a tube catheter with a plunger to deploy the outer frame; a tube catheter with a plunger gripper to deploy the turbine; a gripper catheter to retrieve the turbine.

A cutter catheter can be used to cut the electric cable from the frame when the pump has served its purpose and is no longer needed.

Two embodiments, in accordance with the present invention, are shown in the drawings.

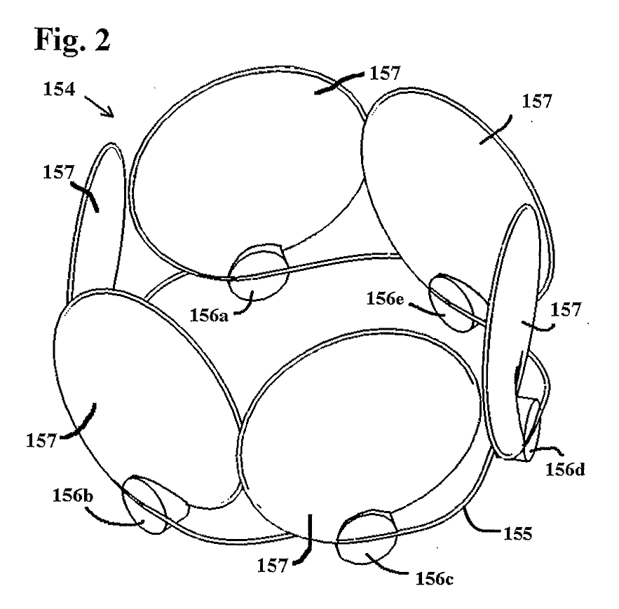

FIG. 2 illustrates a deployable wire turbine for a deployable blood flow enhancing device in accordance with a preferred embodiment of the present invention. The turbine is built from a metal wire 155 shaped to form a ring with six vertical round loops. Six magnets 156a, 156b, 156c, 156d, 156e are attached to the wire to all around the wings, with poles of the facing radially outwards and inwards. Thin polymer membranes 157, are stretched over the six wire loops, and form the surfaces of the blades of the turbine.

The embodiment in FIGS. 1 and 2 shows a turbine built from a single wire frame with attached magnets. The blades are thin film membranes that may be created by dipping the wire loops in a polymer. An advantage of the embodiment of FIG. 2 is that in can be fabricated in very small sizes, and folded down to a very small profile.

FIG. 3 illustrates a deployable blood flow enhancing device in accordance with a preferred embodiment of the present invention, the deployable turbine seen inside. shows a different embodiment of the pump. The pump has two major parts: the outer frame 151 and the propeller turbine 164. The frame 151 is built as a wire frame from metal wires 153, in a tubular shape. Mounted on the frame are six electric coils 152a, 152b, 152c, 152e, (only five coils are visible in this figure, as one is hidden behind the device). The coils are mounted around the frame. The frame is deployed inside a blood vessel and expanded, so that the tubular frame is in contact with the tubular walls of the vessel. The turbine 164 is placed inside the frame. The frame is narrower in the distal and proximal ends, than it is in the middle, so that the turbine will not fall out. The turbine is built from a metal wire. Six magnetic elements (only two magnets 165a, 165b are visible in this figure) are attached to the wire. Six thin triangular metal plates (only four plates 166 are visible in this figure) are mounted on elastic stubs coupled to the magnets, and form the surfaces of the blades of the turbine. When the turbine is inside the frame the coils and the magnets act as an electric motor that rotates the turbine. When the pump is activated, current running through the coils rotates the turbine and pushes blood forward. The forces exerted by the rotational motion of the turbine inside the blood fold the blades inwardly. The blades and the metal stubs they are mounted on are elastic, so when the rotation stops they fold outwards, leaving the lumen of the pump open for blood to flow freely.

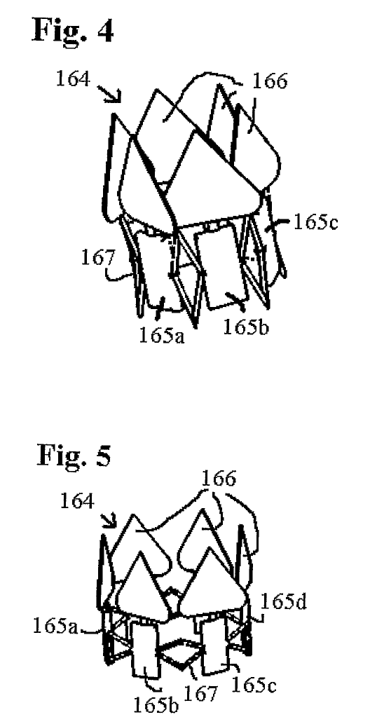

FIG. 4 illustrates an alternative embodiment for a deployable turbine for a blood flow enhancing device in accordance with a preferred embodiment of the present invention, in a contracted state. The turbine is built from a metal wire 167. Six magnets (only three magnets 165a, 165b, 165c are visible in this figure) are attached to the wire to all around, with poles of the facing radially outwards and inwards. Six thin triangular metal plates 166 are mounted on elastic stubs connected to the magnets, and form the surfaces of the blades of the turbine.

FIG. 5 illustrates the deployable turbine shown in FIG. 4 in a deployed state. The turbine is built from a metal wire 167. Six magnets (only four magnets 165a, 165b, 165c, 165f are visible in this figure) are attached to the wire to all around, with poles of the facing radially outwards and inwards. Six thin triangular metal plates 166 are mounted on elastic stubs connected to the magnets, and form the surfaces of the blades of the turbine. The blades are provided with a slight lateral twist, with the attack edge leaning into the direction of rotation. The forces exerted on the blades cause the blades to fold inwardly, forming the propeller. When in the folded state, the propeller, when rotating, forces the blood forward. The elasticity of the stubs can be set to the proper level to determine when the blades will give in to the pressure and fold inwardly. This mechanism is also employed in the design of the embodiment shown in FIG. 2. There the elasticity of the flaps is attributed to the wire formation, and the flaps are slightly twisted as a result of the looped design of the wire.

FIG. 6 illustrates the deployable turbine shown in FIG. 5 with the flaps in operational orientation (folded state). The revolution pushes the blood forward, and the backpressure folds the blades inwards. The turbine is built from a metal wire 167. Six magnets 165a, 165b, 165c, 165d, 165e, 165f (only five magnets 165a, 165b, 165c, 165d, 165f are visible in this figure) are attached to the wire to all around, with poles of the facing radially outwards and inwards. Six thin triangular metal plates 166 are mounted on elastic stubs connected to the magnets, and form the surfaces of the blades of the turbine.

The embodiment shown in FIGS. 3,4,5 and 6 shows a set of magnets connected by wires with triangular metal blades mounted on the magnets. The blades are slightly twisted and when the turbine revolves, they act like a screw and advance blood forward.

FIG. 7 illustrates a deployable blood flow enhancing device in accordance with a preferred embodiment of the present invention, with a control unit.

The coils are connected through lead wires to a thin electric cable 59 that connects the frame 151 with a controller box 58 that houses a power source (like a battery, for example, or a rechargeable unit) and an electrical circuit that generates the alternating current to the coils to drive the pump. This electric cable also may also act as a guide wire for the catheters that deploy and retrieve the propeller. The controller box can be implanted inside the body or kept outside the body. The electric cable 59 leads from the controller box to the pump, along the vascular system. The controller box may include a computer, and may be programmed to run the pump in different intervals and regimes. The major parts of the pump, the frame 151 and the turbine 164, are placed inside a patient's blood vessel, to increase blood flow in that vessel. The controller box is kept outside the patient's body, or is implanted under the patient's skin. The proximal end of the cable is attached to the controller box. The cable is threaded through the vascular system and leads to the pump. The distal end of the cable is attached to the frame of the pump, and the lead wires in the cable lead to the coils mounted on the frame.

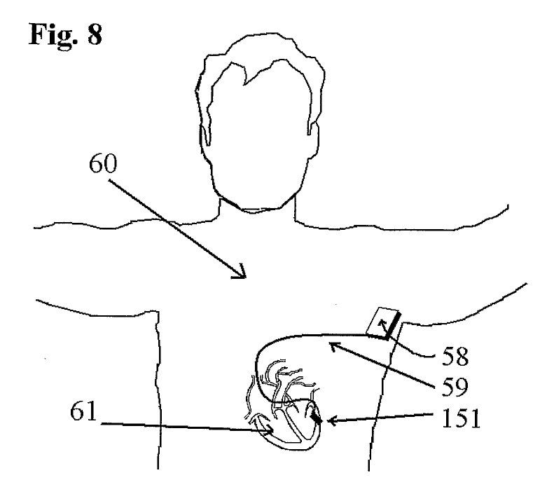

FIG. 8 illustrates a deployable blood flow enhancing device in accordance with a preferred embodiment of the present invention, with a control unit, implanted in a patient. The device is implanted in a patient with the controller box implanted under the patient's skin near his armpit, the lead wire extended to the heart (where the device is positioned in this example) through the vascular system. The device is deployed in the heart 61 of a patient 60. The frame 151 is deployed in a coronary blood vessel (artery or vein), and the lead wire 59 is tracked through the vascular system to the controller box 58, close to the patient's armpit.

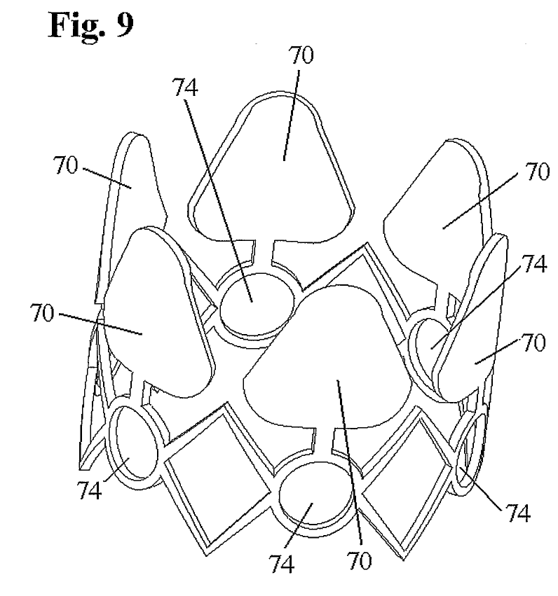

FIGS. 9-12 describe how to make a turbine by a laser cutting a metal tube and shaping it to form a turbine. This manufacturing method can save time and money, since most of the device is cut out from a single part. The only additional parts that have to be attached are the magnets since they are made from a different material.

FIG. 9 illustrates a first stage in the manufacturing process of a deployable turbine for a deployable blood flow enhancing device in accordance with a preferred embodiment of the present invention. This initial shape is cut out of a thin wall of a Nitinol tube using standard laser cutting technique known in cutting stents. This single piece metal structure is the frame of the turbine with the blades 70, and the mounting sockets for the magnets 74 already integral to it.

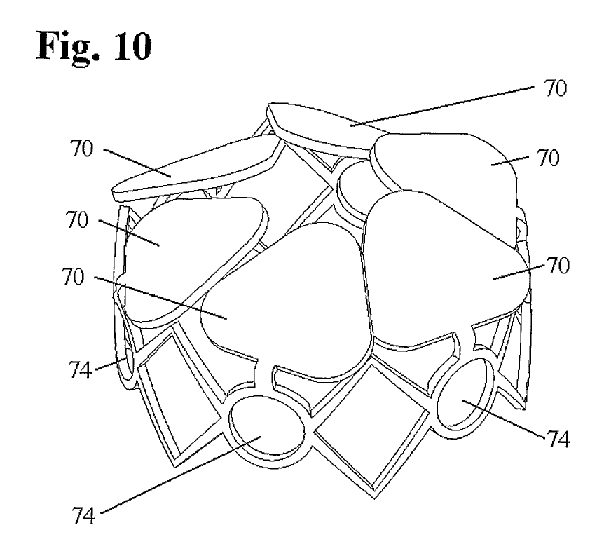

FIG. 10 illustrates a second stage in the manufacturing process of a deployable turbine for a deployable blood flow enhancing device in accordance with a preferred embodiment of the present invention, where the flaps are press-folded to form a turbine. The tubular shape is press-folded to form the shape of the turbine and than heat-treated to retain this shape. Nitinol is a shape memory Nickel Titanium alloy and will elastically come back to this shape when deployed even though for delivery the blades will be straightened and the tube will be crimped down to a low profile, and delivered through a narrow tube.

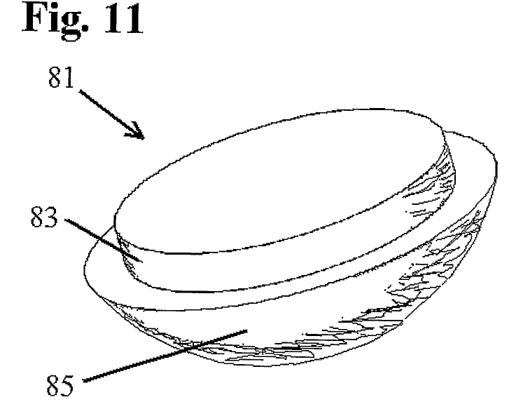

FIG. 11 illustrates a magnetic element 81 to be incorporated with a deployable turbine for a blood flow enhancing device in accordance with a preferred embodiment of the present invention. A small magnetic element with a tubular portion 83, shaped to fit into the sockets in the frame of the turbine, and a spherical portion 85, shaped to engage and slide on the outer frame of the device that serves as the stator of the electric motor.

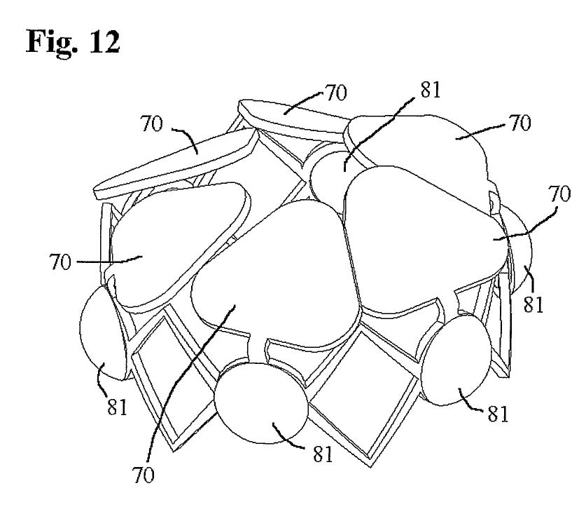

FIG. 12 illustrates a turbine propeller with embedded magnetic elements. The magnets 81 are mounted on the turbine frame and magnetized. This is the complete assembly of the turbine, with the blades 70 forming the propeller shape.

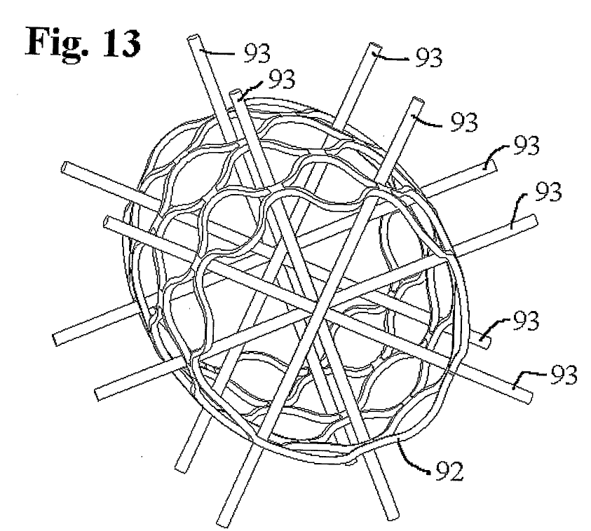

FIGS. 13 to 17 illustrate the steps of putting the electric motor coils on a coronary stent.

FIG. 13 illustrates a first step in incorporating electric motor coils with a stent. To wind the electric coils on the stent 92 frame pins 93 are inserted through the struts of the stent. These pins serve as a temporary scaffold for winding the coils.

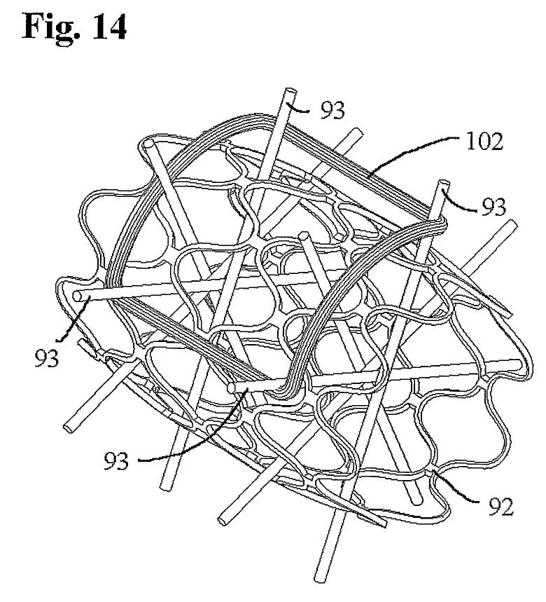

FIG. 14 illustrates a second step in incorporating electric motor coils with a stent. A Coil 102 is created by winding a thin metal wire around several of the pins 93, on one side of the stent 92. The winding is repeated for as many turns as needed to create the coil. The two ends of the thin wire are left dangling and would later be attached by a cable of the electronic circuit.

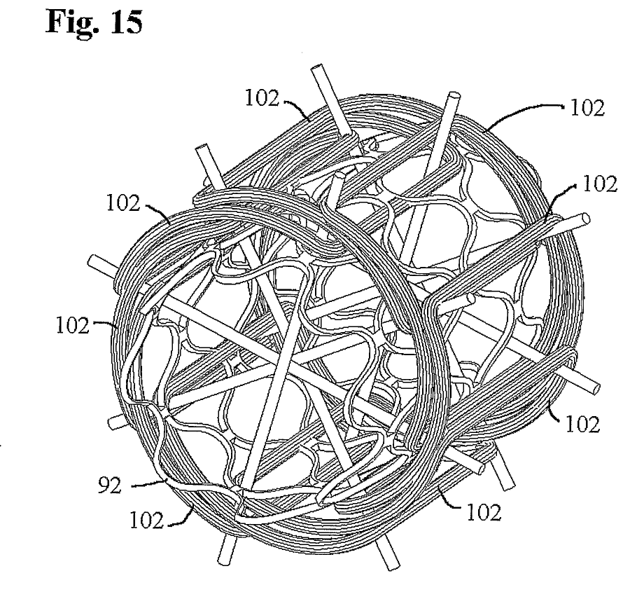

FIG. 15 illustrates a third step in incorporating electric motor coils with a stent. More coils 102 are created by winding the wire around the pins on all sides of the stent 92. The coils are placed overlapping each other much like a conventional electric motor.

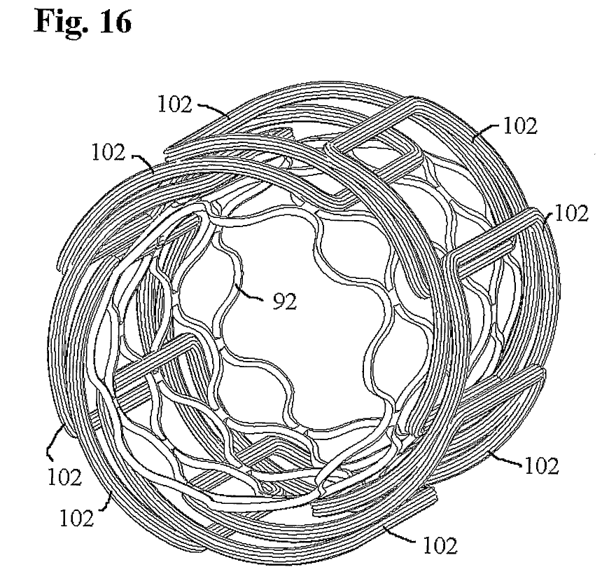

FIG. 16 illustrates a fourth step in incorporating electric motor coils with a stent. The pins are removed and leave the stent 92 covered with the coils 102.

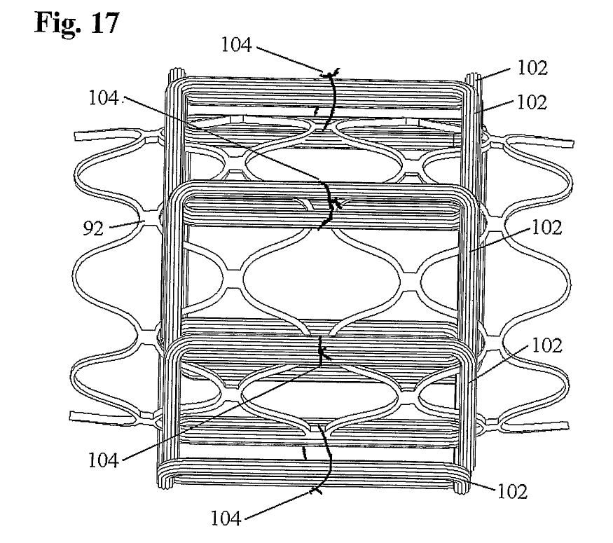

FIG. 17 illustrates a fifth step in incorporating electric motor coils with a stent. The coils 102 are fixed to the stent frame by tying them with sutures 104. The sutures are place on two sides of each coil, all around the centerline of the stent, but leaving the coils free to fold, when the stent is crimped to its low delivery profile.

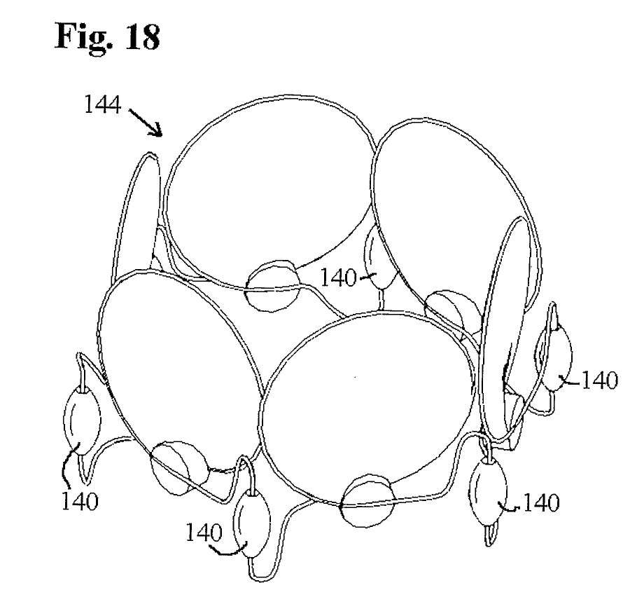

FIG. 18 illustrates the wire turbine shown in FIG. 2 with beads threaded on the wire. This is the wire turbine of FIG. 2, with beads 140 threaded on the wire. The beads are placed on longitudinal sections of the wire and act as ball bearings. These bearings can make contact with the outer stent frame and roll over it with minimal friction, allowing the turbine to revolve freely.

It should be clear that the description of the embodiments and attached Figures set forth in this specification serves only for a better understanding of the invention, without limiting its scope.

It should also be clear that a person skilled in the art, after reading the present specification could make adjustments or amendments to the attached Figures and above described embodiments that would still be covered by the present invention.

1.-16. (canceled)

17. An implantable stent adapted for fluid pumping in a lumen of the body having a wall, comprising:
(a) an expandable tubular structure expandable in the lumen against said wall to form a conduit anchored to the wall; and
(b) at least one conducting coil configured to deliver electromagnetic field in the lumen responsive to electric current supplied to the coil.

18. A stent according to claim 17, further comprising an element inside the stent conduit wherein the electromagnetic field is adapted to impart force on at least a part of the element.

19. A stent according to claim 18, wherein the force moves the element in at least one of rotation and translation movement in the stent conduit.

20. A stent according to claim 17, wherein the lumen is a coronary blood vessel.

21. An implantable electric pump fitting into a lumen of a body having a wall and adapted to enhance liquid flow, comprising:
(a) an expandable tubular structure expandable in the lumen against said wall to form a conduit anchored to the wall, comprising at least a part of an electric motor stator; and
(b) a movable element inside the conduit, comprising at least a part of an electric motor rotor.

22. A pump according to claim 21, wherein the element comprises at least one expandable member.

23. A pump according to claim 21, wherein the element is configured for later implantation inside the conduit of the structure after expansion thereof.

24. A pump according to claim 21, wherein the element is configured for removal by a catheter after implantation.

25. A pump according to claim 21, wherein the element comprises a plurality of folding blades adapted to impel liquid.

26. A pump according to claim 25, wherein the blades are foldable in a configuration adapted to allow liquid passage in the conduit when the motor is off.

27. A pump according to claim 21, wherein the lumen is a coronary blood vessel.

28. A stent configured to propel blood unidirectionally in a blood vessel having a wall, comprising:
(a) an expandable tubular structure expandable in the vessel against said wall to form a conduit anchored to the wall; and
(b) a moveable element inside the conduit, adapted to propel blood in one direction.

29. A stent according to claim 28, wherein the element moves along the conduit.

30. A stent according to claim 29, wherein the movement is electrically powered.

31. A stent according to claim 28, wherein the blood vessel is a coronary blood vessel.

32. A therapeutic system comprising:
(a) a blood flow enhancing device comprising a pump;
(b) a heart stimulating device;
(c) a control circuitry; and
(d) a power supply.

33. A system according to claim 32, wherein the control circuitry controls the operation of at least one of the blood flow enhancing device and heart stimulating device.

34. A system according to claim 32, wherein the blood flow comprises blood flow in a coronary vessel.

35. A system according to claim 32, wherein the heart stimulating device is a pace maker.

36. A method for forming a coil on a tubular structure having holes, comprising:
(a) flattening the structure, at least partially;
(b) temporarily inserting pins into at least a part of the holes;
(c) turning a wire around the pins; and
(d) expanding the structure back to the tubular form.

37. A method for fabrication a miniature impeller, comprising:
(a) providing a tube; and
(b) cutting the tube with at least one contour of at least a part of the impeller.

38. A method according to claim 37, wherein the tube comprises a shape memory alloy.

39. A method according to claim 37, wherein the cutting is carried out by laser.
